(19) United States
(12) Patent Application Publication
(10) Pub. No.: US 2009/0304797 A1
(43) Pub. Date: Dec. 10, 2009

(54) Process for the Preparation of Micronized Valsartan

(76) Inventors: Venkatasubramanian Radhakrishnan Tarur, Mumbai (IN); Dhananjay Govind Sathe, Thane (IN); Narayana Rao, Mumbai (IN); Kamles Digambar Sawant, Mumbai (IN); Sasi Kumar Mohan Thoovara, Mumbai (IN); Bhupendra Shaligram Rane, Dombivli (IN)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 12/304,148

(22) PCT Filed: Jun. 22, 2007
(86) PCT No.: PCT/IN07/00249

§ 371 Date: Feb. 2, 2009

(30) Foreign Application Priority Data
Jun. 23, 2006 (IN) 985/MUM/2006

(51) Int. Cl. A61K 9/14; C07D 257/04; A61K 31/41; A61P 9/12

(52) U.S. Cl. 424/489; 548/253; 514/381

(57) ABSTRACT

The present invention relates to process for preparing micronized Valsartan with particle size distribution of d₁₀ less than 5μ, d₅₀ less than 10μ and d₉₀ less than 20μ preferably d₉₀<10μ.

### PRIORITY

This application claims the benefit of Indian Provisional Application No. 985/MUM/2006 filed on Jun. 23, 2006.

### TECHNICAL FIELD

The present invention relates to stable micronized Valsartan and process for preparation thereof.

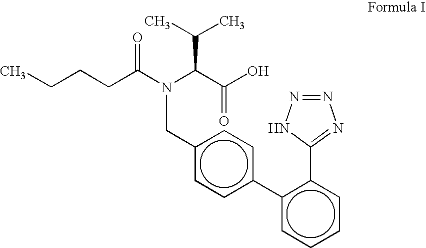

### BACKGROUND & PRIOR ART

Valsartan which is chemically known as N-(1-Oxopentyl)-N-[[2′-(1H-tetrazol-5-yl)[1,1′-biphenyl]-4-yl]methyl]-L-valine [Formula 1] is an antihypertensive agent. Synthesis of Valsartan is disclosed in various references, including U.S. Pat. No. 5,399,578, U.S. Pat. No. 5,965,592, U.S. Pat. No. 6,271,375, U.S. Pat. No. 5,260,325, WO01/082858, WO02/006253, WO97/30036, WO99/67231, Moenius et al., Int. J. Labeled compounds and radiopharmaceuticals, 43(13), 1245-1252 (2000) and Peter et al., Bioorganic & Med. Chem. Lett., 4(1), 29-34 (1994).

Valsartan is an active angiotensin II antagonist specifically acting on the AT1 receptor sub type. U.S. Pat. No. 5,399,578 discloses use of Valsartan as an antihypertensive agent. U.S. Pat. No. 6,395,728 discloses use of Valsartan in the treatment of diabetes related hypertension. U.S. Pat. No. 6,465,502 and U.S. Pat. No. 6,485,745 discloses use of valsartan in the treatment of lung cancer.

U.S. Pat. No. 5,399,578 discloses crystallization of Valsartan from ethyl acetate (m.p. 105-115° C.). Merck Index 13^{th} Edition and U.S. Pat. No. 5,399,578 reports crystallization of Valsartan from diisopropyl ether (m.p. 116-117° C.). Int. J. Labeled compounds and Radiopharmaceuticals, 43 (13), 1245-1252 (2000) reports crystallization of Valsartan from 1:1 mixture of ethyl acetate:hexane.

WO04/083192 discloses process to prepare highly pure amorphous Valsartan along with eleven crystalline polymorphic forms. WO03/089417 also discloses two crystalline polymorphic forms of Valsartan.

WO 04/087681 discloses a process for preparation of amorphous form of Valsartan, which involves spray drying method using alcoholic solution of Valsartan. The patent application discloses a process for preparation of amorphous form but does not disclose the particle size distribution.

Solid state properties of a drug can have a great influence on the solubility of the drug. Particle size is a very critical physical parameter. Generally, the bioavailability of an API is inversely proportional to the particle size. Reduced particle size increases bioavailability of insoluble API's (active pharmaceutical ingredient). The rate of dissolution of drug in the stomach fluid has a therapeutic consequence as it imposes an upper limit on the rate at which the orally administered drug can reach the blood stream of the patient.

The rate of dissolution especially for water insoluble drug substance can be improved and a stable dissolution profile can be obtained by reduced particle size. The reduction in the particle size will improve the rate of dissolution, which will sequentially improve the bioavailability of the drug substance.

The conventional method of micronization of API's involve jet or fluid energy mills and ball mill techniques. The basic principle in all these techniques involve application of force on the particle in the form of collision which acts at the imperfection in crystal surface, initiating crack propagation through the particle. As the size of the particle decreases, the number of imperfections decrease thereby the task of further reducing the particle size becomes more difficult.

Drawback of micronization using jet mill is that there is a friction amongst particles and between particles and the mill surface. It can be of concern if the material to be milled is abrasive thus causing considerable damage to the mill surface and can also result in contamination of the API. The method involves the collision of the solid particles, which will generate heat, which may result in thermal decomposition thereby rendering the technique unsuitable for heat sensitive compounds. The heat generated during micronization can also lead to change in the physical appearance and/or polymorphic form.

When Valsartan is crystallized by using ethyl acetate, as taught in U.S. Pat. No. 5,399,578, particle size distribution of Valsartan, obtained after passing through 30 mesh sieve, is as follows: d₁₀=5.039μ, d₅₀=77.29μ and d₉₀=383.55μ.

Hence there is a need in the art for stable micronized Valsartan amorphous form and process for producing the same. The present invention provides a process for preparing micronized Valsartan having particle size d₉₀<20 microns, without affecting its physical form or appearance, which has greater bioavailability than crystalline forms and may be more suitable for formulation of an active pharmaceutical ingredient.

Thus, the present invention is aimed to provide Valsartan with improved solid state characteristics or properties, especially particle size without affecting its chemical activity and physical appearance, by a simple and economical process thereby overcoming the problems associated with the prior art processes.

### OBJECT OF THE INVENTION

The object of the present invention is to provide stable micronized Valsartan with particle size of d₉₀<20μ.

Another object of the present invention is to provide a process for preparing stable micronized Valsartan with a particle size d₉₀<20μ.

Yet another object of the present invention is to provide a process for preparing stable micronized Valsartan, without affecting its physical appearance.

Yet another object of the present invention is to provide stable micronized Valsartan with improved dissolution profile and enhanced bioavailability.

Further object of the present invention is to provide pharmaceutical composition comprising stable micronized Valsartan having particle size d₉₀<20μ, preferably d₉₀<10μ.

### SUMMARY OF THE INVENTION

The present invention discloses stable micronized Valsartan having particle size d₉₀<20μ Preferably, the invention discloses micronized Valsartan with particle size distribution, d₁₀<5μ, d₅₀<10μ and d₉₀<20μ, preferably d₉₀<10μ.

The present invention provides a process for preparation of stable micronized Valsartan with particle size d₉₀<20μ, preferably d₁₀<5μ, d₅₀<10μ, d₉₀<20μ, more preferably d₉₀<10μ.

According to one aspect of the invention, there is provided a process for the preparation of stable micronized Valsartan comprising crystallization of Valsartan with suitable lower chlorinated hydrocarbon preferably methylene dichloride to provide micronized Valsartan with particle size d₉₀ less than 20μ.

According to another aspect of the invention, there is provided a process for the preparation of stable micronized Valsartan which involves spray drying.

The invention further encompasses pharmaceutical compositions comprising stable micronized Valsartan having particle size d₉₀<20μ with pharmaceutically acceptable excipients.

### BRIEF DESCRIPTION OF THE FIGURES

FIG. 1 illustrates the XRPD of stable micronized Valsartan obtained by crystallization.

FIG. 2 illustrates the DSC thermogram of stable micronized Valsartan obtained by crystallization.

FIG. 3 illustrates the XRPD of stable micronized Valsartan obtained by spray drying method.

FIG. 4 illustrates the DSC thermogram of stable micronized Valsartan obtained by spray drying method.

### DETAILED DESCRIPTION OF THE INVENTION

The present invention describes a process to prepare stable micronized Valsartan having particle size d₉₀<20μ. Valsartan being insoluble in water, particle size reduction can improve the dissolution profile and hence drug bioavailability. The advantage of the process of the present invention is that it directly gives Valsartan with particle size d₉₀<20μ without involving milling and thus does not affect the physical appearance of the end product.

Particle size can affect the solubility properties of a compound. Particle size reduction is one of the methods to overcome the solubility issue of poorly soluble APIs like Valsartan. Particle size reduction can also improve a compound's dissolution rate, and hence, its bioavailability. The enhanced dissolution rate is achieved with the increase in the surface area as a result of particle size reduction. Sometimes the rate of dissolution of a poorly soluble drug is the rate limiting factor in its rate of absorption in the body. However, these drugs may be more readily bioavailable if administered in a finely divided state. Particle size may affect the flowability of the API which has consequences in the production process of pharmaceutical products.

Conventional methods of particle size reduction, such as comminution rely upon mechanical stress to disaggregate the active compound. The principal operations of conventional size reduction are milling of a feedstock material and sorting of the milled material by size. A fluid energy mill, or micronizer, is an especially preferred type of mill for its ability to produce particles of small size in a narrow size distribution. The critical parameters of comminution are well-known to the industry, thus permitting an efficient, reproducible and economic means of particle size reduction. However, the mechanical forces inherent to comminution, such as milling and grinding, often impart significant amounts of physical stress upon the drug product which may induce degradation. The thermal stress which may occur during comminution and spray drying is also a concern when processing thermo-sensitive or unstable active compounds. Moreover, traditional comminution and micronizing techniques may not be able to reduce particle size sufficiently to significantly improve bioavailability or permeability.

The present invention thus provides a process for producing stable micronized Valsartan with particle size d₉₀<20μ, preferably d₁₀<5μ, d₅₀<10μ and d₉₀<20μ, more preferably d₉₀<10μ using crystallization. This crystallization technique avoids the conventional techniques used for producing micronized Valsartan. The process for preparing stable micronized Valsartan by crystallization is simple, cost effective and consumes less energy.

The crystallization process for producing stable micronized Valsartan, involves dissolution of Valsartan in lower chlorinated hydrocarbons which results in micronized Valsartan with particle size distribution d₁₀<5μ, d₅₀<10μ and d₉₀<20μ preferably d₉₀<10μ.

Alternatively, a solution of Valsartan in an organic solvent is spray dried. The spray drying process is economical, less energy consuming, can be run continuously leading to higher production, requires less maintenance and is industrially viable.

Valsartan used in the process of the present invention can be prepared from any methods including methods disclosed in the prior art, some of which are incorporated herein by reference.

The term ‘micronized’ as used herein refers to Valsartan with particle size d₉₀<20μ preferably d₁₀<5μ, d₅₀<10μ and d₉₀<20μ, more preferably d₉₀<10μ.

As used herein, a solvent is any liquid substance, which has capacity to dissolve the organic compound Valsartan, either at room temperature or higher.

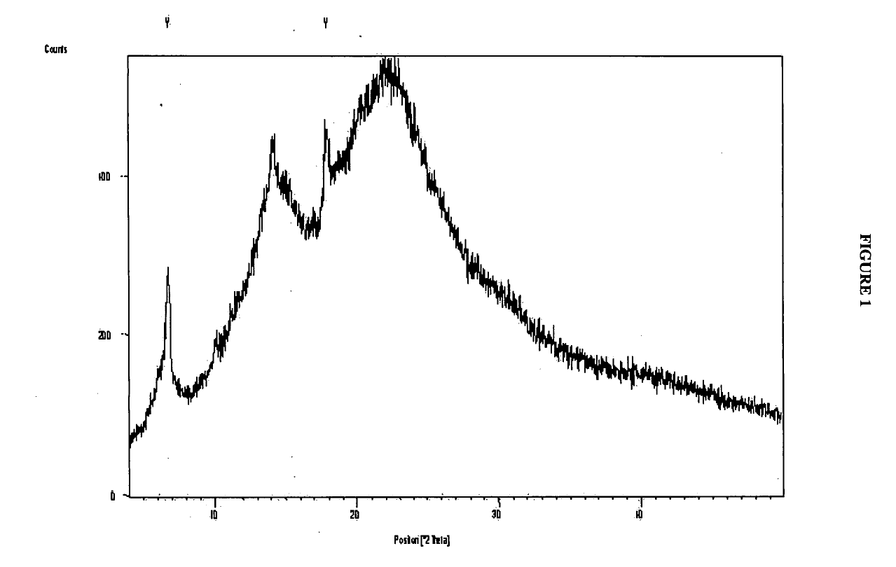

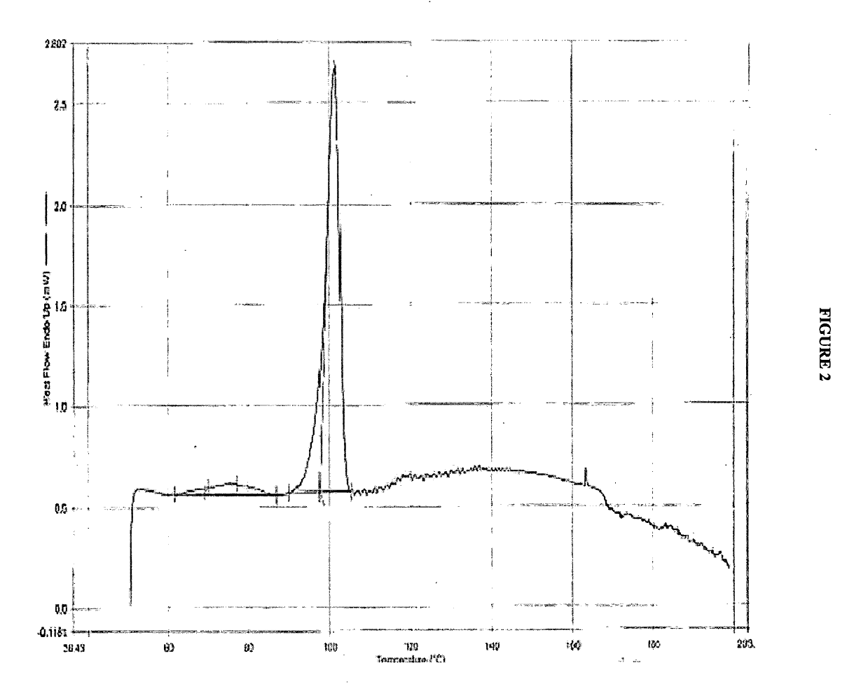

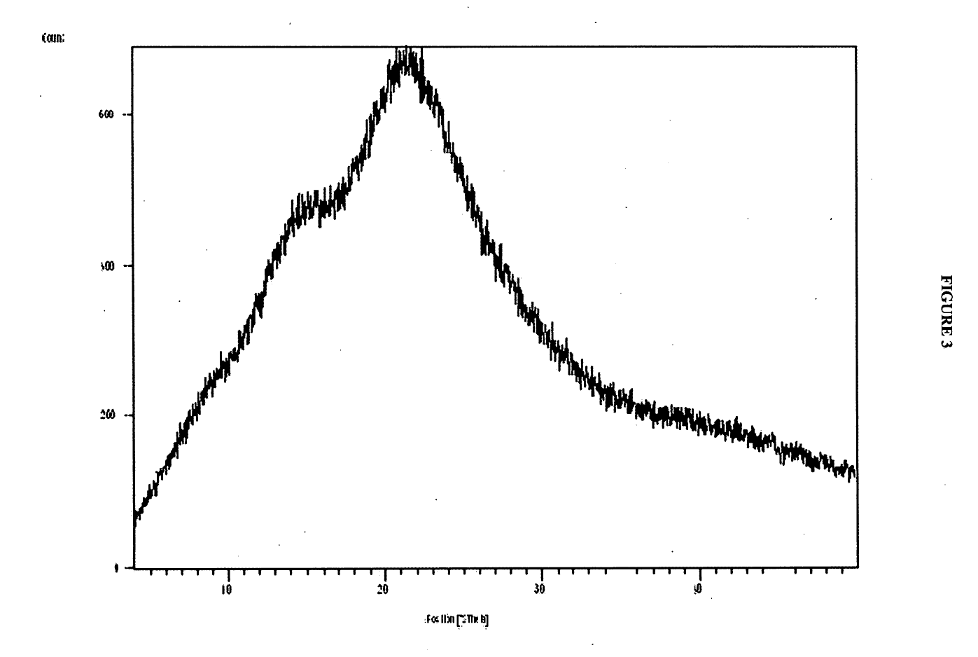

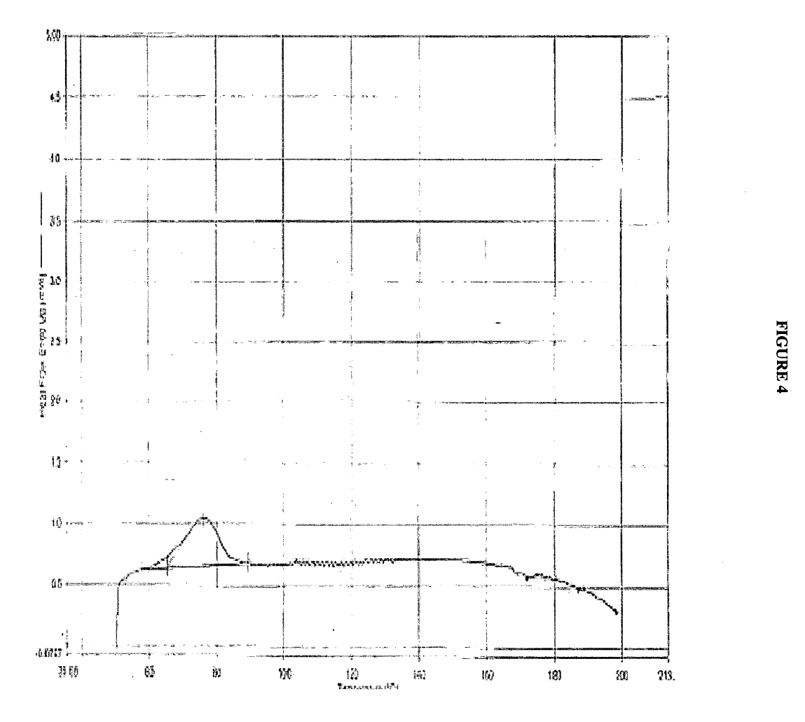

The micronized Valsartan, obtained by the process of the present invention, is amorphous in nature, characterized by X-ray powder diffraction (FIGS. 1 and 3) and DSC (FIGS. 2 and 4).

In a preferred embodiment, the process of the present invention describes the preparation of stable micronized Valsartan comprising of multiple crystallization steps, preferably three, more preferably two to get stable micronized Valsartan. The crystallization process for making micronized Valsartan involves dissolution of crude Valsartan in lower chlorinated hydrocarbons, preferably methylene dichloride.

In the first crystallization step, crude Valsartan is dissolved in chlorinated hydrocarbon such as methylene dichloride at 35-45° C. The solution is cooled to 0-5° C. and the purified Valsartan is isolated by filtration. The solid obtained has a particle size of d₉₀>150μ.

In the second crystallization step, the solid obtained from first step is redissolved in methylene dichloride at 35-45° C. and the obtained solution is filtered. The filtrate thus obtained is cooled to 0-5° C. and the separated solid is isolated by filtration.

The wet cake obtained after above crystallization step is suspended in n-pentane at 10-20° C. and is stirred for one hour. The mixture is filtered and the obtained solid is dried under vacuum at 50° C. The solid obtained has a particle size of d₉₀=15 to 30 before sieving. This solid is passed through 30 mesh to get stable micronized Valsartan of particle size, d₉₀=below 20μ preferably below 15μ, more preferably below 10μ.

Alternatively, micronized Valsartan is prepared by dissolving the crude Valsartan in organic solvents and the solvent is removed by spray drying technique. The spray drying process is economical, less energy consuming, can be run continuously leading to higher production, requires less maintenance and is industrially viable.

The spray drying process for preparing micronized Valsartan comprises dissolving Valsartan in organic solvent at a temperature range of 30-40° C. Concentration of Valsartan solution used for spray drying is about 10% weight/volume. Spray drying is carried out at the inlet temperature range of 40° C. to 140° C., preferably 120° C. and outlet temperature range of 35° C. to 85° C., preferably 65° C. Valsartan obtained after spray drying process has the following particle size distribution, d₁₀<5μ, d₅₀<10μ and d₉₀<20μ preferably d₉₀<10μ.

The organic solvent used is selected from the group consisting of acetone, a mixture of acetone and water, lower aliphatic esters preferably ethyl acetate or chlorinated hydrocarbons preferably methylene dichloride.

The dissolution is carried out at temperature 20° C.-40° C., preferably 30° C.

The concentration of Valsartan used in spray drying is 5-15% preferably 8-12%.

The invention describes pharmaceutical compositions comprising stable micronized Valsartan with fine particle size of d₉₀<20μ, preferably below 15μ, more preferably below 10μ along with pharmaceutically acceptable excipients selected from diluent or pharmaceutically acceptable carriers, fillers, bulking agents, binders, wetting agents, disintegrating agents, surface active agents, lubricants, and the like. The pharmaceutical composition can include at least one diluent or excipient. For the pharmaceutical compositions, various types of administration unit forms can be selected depending on the therapeutic purpose, for example tablets, pills, powders, liquids, suspensions, emulsions, granules, capsules, suppositories, injection preparations (solutions and suspensions), and the like. Any excipient commonly known and used widely in the art can be used in the pharmaceutical compositions described herein.

X-ray powder diffraction pattern has been obtained on Xpert'PRO, Panalytical, diffractometer equipped with accelerator detector using Copper Kα (λ=1.5406 Å) radiation with scanning range between 4-50 2-theta at scanning speed of 2°/min.

Differential Scanning Calorimeter was performed on Mettler DSC 20 instrument. Samples of 2 mg to 3 mg weighed in aluminum crucibles with holes Were scanned at a heating rate of 11° C. per minute under nitrogen atmosphere at rate of 35 ml/min.

While the present invention has been described in terms of its specific embodiments, certain modifications and equivalents will be apparent to those skilled in the art and are included within the scope of the present invention. The examples are provided to illustrate particular aspects of the disclosure and do not limit the scope of the present invention.

### Example 1

### Preparation of Micronized Valsartan

Valsartan, prepared by known process, was dissolved in acetone at a temperature range of 30° C. to 40° C. and was subjected to spray drying. Concentration of Valsartan solution used for spray drying is about 10% weight/volume. Spray drying was carried out at the inlet temperature of 120° C. and outlet temperature of 65° C.

Particle size distribution is as follows: d₁₀=1.906μ, d₅₀=4.134μ, d₉₀=7.554μ

### Example 2

### Preparation of Micronized Valsartan

Valsartan, prepared by known process, was dissolved in acetone containing 10% water at a temperature range of 30-40° C. and was subjected to spray drying. Concentration of Valsartan solution used for spray drying is about 10% weight/volume. Spray drying was carried out at the inlet temperature of 120° C. and outlet temperature of 65° C.

Particle size distribution is as follows: d₁₀=1.686μ, d₅₀=4.420μ, d₉₀=8.684μ

### Example 3

### Preparation of Micronized Valsartan

Valsartan, prepared by known process, was dissolved in ethyl acetate at a temperature range of 30° C. to 40° C. and was subjected to spray drying. Concentration of Valsartan solution used for spray drying is about 10% weight/volume Spray drying was carried out at the inlet temperature of 120° C. and outlet temperature of 65° C.

Particle size distribution is as follows: d₁₀=1.5421μ, d₅₀=3.850μ, d₉₀=7.869μ

### Example 4

### Preparation of Micronized Valsartan

Valsartan, prepared by known process, was dissolved in methylene dichloride at a temperature range of 30° C. to 40° C. and was subjected to spray drying. Concentration of Valsartan solution used for spray drying is about 10% weight/volume. Spray drying was carried out at the inlet temperature of 120° C. and outlet temperature of 65° C.

Particle size distribution is as follows: d₁₀=2.011μ, d₅₀=4.636μ, d₉₀=8.611μ.

### Example 5

### Preparation of Micronized Valsartan by Multiple Crystallization

### A) First Crystallization.

100 g Valsartan (prepared by known process) was dissolved in 2 litre methylene dichloride at a temperature range of 35-45° C. The hot solution was filtered and cooled to 0-5° C. The solution was maintained at same temperature under stirring for 2 hours. The separated solid was filtered.

Particle size distribution is as follows: d₁₀=1.869μ, d₅₀=18.401μ, d₉₀=202μ

### B) Second Crystallization.

Wet Valsartan obtained by first crystallization process was dissolved in 20 volumes of methylene dichloride at a temperature range of 35° C. to 45° C. The hot solution was filtered through a micron filter paper [pore size: 0.5μ or 1μ]. The filtrate was cooled to 0-5° C. The solution was maintained at same temperature under stirring for 2 hours. The separated solid was filtered. The wet solid was suspended in 20 ml of n-pentane at 10° C. to 20° C. and stirred for 1 hour. The solid was filtered and dried under vacuum at 50° C. The solid obtained was passed through 30 mesh to get the stable micronized Valsartan. Particle size distribution is as follows: d₁₀=0.71μ, d₅₀=2.687μ, d₉₀=8.42μ

### Example 6

Stable micronized Valsartan tablets 320 mg with particle size distribution d₉₀ less than 10 microns.

Slugging/Deslugging: Valsartan and microcrystalline cellulose were weighed, sifted and mixed together. The mixture was then lubricated with crospovidone, colloidal silicon dioxide and magnesium stearate. The blend was slugged using bigger punches, milled (deslugged) to size, then again lubricated with crospovidone, colloidal silicon dioxide and magnesium stearate. The final blend obtained was further compressed to yield tablets. The tablets were then coated.

As the material was fine, direct compression was not possible hence the method followed was slugging/deslugging.

### Dissolution Profile:

Dissolution medium: 0.067M phosphate buffer pH 6.8. Dissolution was conducted using USP Apparatus II.

Valsartan: Particle size- d₉₀: 6.170 microns

| Sr. no. | Time in minutes | % Drug Release |
|---|---|---|
| 1. | 10 | 85.21 |
| 2. | 15 | 95.27 |
| 3. | 20 | 97.95 |
| 4. | 30 | 98.94 |
| 5. | 45 | 99.14 |

Stable micronized Valsartan obtained by the process of the present invention complies with the absorbance test, described in USP.

1. A process for preparation of the Valsartan of claim 19 comprising the steps of:
1. dissolving crude Valsartan in a suitable organic solvent;
2. filtering the solution and cooling the filtrate to 0-5° C. followed by stirring;
3. isolating the solid material from the cooled solution;
4. dissolving the obtained solid material in suitable organic solvent at a temperature range of 35-45° C.;
5. filtering the solution through micron filter and cooling the filtrate to 0-5° C. followed by stirring to obtain a solid material;
6. slurrying the obtained solid in C₅-C₇ aliphatic hydrocarbon; and
7. filtering the solid obtained, drying under vacuum at 50° C. and passing through 30 mesh sieve to get micronized Valsartan.

2. The process as claimed in claim 1, wherein the micronized Valsartan obtained is amorphous in nature.

3. The process as claimed in claim 1, wherein the micronized Valsartan obtained has particle size of d₉₀ less than 10μ.

4. The process as claimed in claim 1, wherein the suitable organic solvent is selected from a group consisting of chloroform and methylene dichloride.

5. The process as claimed in claim 1, wherein pore size of the micron filter is 0.5 to 3.5μ.

6. The process as claimed in claim 1, wherein C₅-C₇ aliphatic hydrocarbon is n-pentane.

7. A process for preparation of the Valsartan of claim 19 comprising,
a) dissolving crude Valsartan in a suitable organic solvent; and
b) removal of solvent by spray drying.

8. The process as claimed in claim 7, wherein suitable organic solvent is selected from acetone, mixture of acetone and water, lower aliphatic esters or chlorinated hydrocarbons.

9. The process as claimed in claim 7, wherein concentration of Valsartan solution used in spray drying is 5-15%.

10. The process as claimed in claim 7, wherein Valsartan solution is spray dried at the inlet temperature range of 40 to 140° C.

11. The process as claimed in claim 7, wherein Valsartan solution is spray dried at the outlet temperature range of 35-85° C.

12. The process as claimed in claim 1 wherein the micronized Valsartan has particle size distribution d₁₀<5μ, d₅₀<10μ and d₉₀<20μ.

13. The process as claimed in claim 12 wherein the micronized Valsartan has particle size distribution d₁₀<3μ, d₅₀<5μ and d₉₀<10μ.

14. (canceled)

15. (canceled)

16. (canceled)

17. The process as claimed in claim 7 wherein the micronized Valsartan has particle size distribution d₁₀<5μ, d₅₀<10μ and d₉₀<20μ.

18. The process as claimed in claim 17 wherein the micronized Valsartan has particle size distribution d₁₀<3μ, d₅₀<5μ and d₉₀<10μ.

19. Valsartan having particle size d₉₀ less than about 20μ.

20. The valsartan of claim 19, having particle size d₅₀ less than about 10μ and particle size d₁₀ less than about 5μ.

21. The valsartan of claim 20, made by the process of claim 1.

22. The valsartan of claim 20, made by the process of claim 7.

23. The valsartan of claim 19, having particle size d₉₀ less than about 10μ.

24. The valsartan of claim 23, having particle size d₅₀ less than about 5μ and particle size d₁₀ less than about 3μ.

25. The valsartan of claim 24, made by the process of claim 1.

26. The valsartan of claim 24, made by the process of claim 7.

27. A composition of matter comprising the Valsartan of claim 19 together with a pharmaceutically-acceptable excipient.
